(19) United States
(12) Patent Application Publication
(10) Pub. No.: US 2018/0042578 A1
(43) Pub. Date: Feb. 15, 2018

(54) AUTOMATED ULTRASOUND IMAGE MEASUREMENT SYSTEM AND METHOD

(71) Applicant: Carestream Health, Inc., Rochester, NY (US)

(72) Inventors: Ajay Anand, Rochester, NY (US); Zhimin Huo, Pittsford, NY (US)

(21) Appl. No.: 15/474,075

(22) Filed: Mar. 30, 2017

Related U.S. Application Data
(60) Provisional application No. 62/374,022, filed on Aug. 12, 2016.

(51) Int. Cl. A61B 8/08; A61B 8/00

(52) U.S. Cl. CPC A61B 8/5215; A61B 8/4483; A61B 8/4405; A61B 8/461

(57) ABSTRACT

An ultrasound method obtains a desired view and/or orientation of the anatomy of interest for an exam using a transducer. The ultrasound image is captured according to a measurement protocol for the exam. An automated measurement tool is initiated according to sensing of an operator action. A measurement from a region of interest of the desired view is obtained. The measurement or display is concluded according to a sensed operator action.

### CROSS REFERENCE TO RELATED APPLICATIONS

This application claims the benefit of U.S. Provisional Application No. 62/374,022, filed Aug. 12, 2016, entitled AUTOMATED ULTRASOUND IMAGE MEASUREMENT SYSTEM AND METHOD, in the name of Ajay Anand et al., which is hereby incorporated by reference in its entirety.

### FIELD OF THE INVENTION

The invention relates generally to the field of medical diagnostic ultrasound systems and methods, and in particular to a system, method, and interface for automated ultrasound image measurements.

### BACKGROUND

Ultrasound imaging systems/methods are well known. See for example U.S. Pat. No. 6,705,995 (Poland) and U.S. Pat. No. 5,370,120 (Oppelt).

US 2013/0225999 (Banjanin) is directed to gesture commands user interface for ultrasound imaging systems.

US 2011/0251483 (Razzaque) is directed to image annotation in image-guided medical procedures. Images may be annotated during a medical procedure. For example, an image annotation session may be initiated and/or terminated by the operator performing a key stroke, issuing a command (such as a verbal command), performing a gesture with a medical device or hand, pressing a button on the medical device, pressing a foot pedal, pressing a button on the medical device (e.g., a button on a Wacom pen), and the like.

All of the above-identified references are incorporated herein by reference in their entirety.

This disclosure relates to medical diagnostic ultrasound imaging systems, and, more particularly, to a system and method for automated ultrasound image measurement.

For improved accuracy in diagnosis of a patient's condition, it is often useful to be able to obtain quantitative dimensional data for the anatomy of interest. Digitization of ultrasound image content helps to make it possible to obtain metrics for distance, circumference, and other values directly from the image content. In diagnostic assessment, measured dimensional values for a particular patient can be compared against measured values obtained for a broad patient population. Results obtained can help to guide treatment and monitoring of the patient.

While the task of dimensional measurement itself is often straightforward and can be automated for many types of images, there are challenges to acquiring the needed measurement data from ultrasound images in each particular case. For fetal biometrics in particular, it can be difficult for the sonographer to acquire a standardized view, with proper orientation of the subject to allow accurate measurement of features such as head circumference, femur length, and organ size. Other difficulties relate to image quality, which can vary significantly from one exam to the next, complicating the task of properly defining the boundaries of various features to be measured. Workflow challenges that present themselves include the capability to identify one or more optimal views of a region of interest and to save these views without interrupting the ultrasound scanning sequence.

Thus, it can be seen that there would be benefits to methods and apparatus that improve sonographer workflow for acquiring image measurement data.

### SUMMARY

Certain embodiments described herein address the need for improved workflow for obtaining measurements from ultrasound imaging, including offering the advantages of optional computer-assisted processing.

These aspects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to an embodiment of the present disclosure, there is provided an ultrasound method, comprising: using a transducer, obtaining a desired view and/or orientation of the anatomy of interest for an exam; optionally, capturing the ultrasound image according to a measurement protocol for the exam; initiating an automated measurement tool according to sensing of an operator action and obtaining a measurement from a region of interest of the desired view; and concluding measurement or display according to a sensed operator action.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 illustrates a sonographer using an exemplary ultrasound system.

FIG. 3 shows a schematic of an exemplary ultrasound system.

FIG. 4 shows a displayed ultrasound image.

FIG. 5 shows a sequence for dimensional measurement that generally applies for a number of different ultrasound systems.

FIG. 6 is a schematic diagram that shows an ultrasound system with a number of operator interface mechanisms for command entry.

FIGS. 7A and 7B show examples of ultrasound displays showing dimensional metrics.

FIG. 8 is a logic flow diagram showing a sequence for computer-assisted measurement in an ultrasound exam.

FIG. 9 shows an alternate workflow for a computer-assisted measurement method.

### DETAILED DESCRIPTION OF EXEMPLARY EMBODIMENTS

The following is a detailed description of the embodiments of the invention, reference being made to the drawings in which the same reference numerals identify the same elements of structure in each of the several figures.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “subject” or “body” or “anatomy” is used to describe a portion of the patient that is undergoing ultrasound imaging. The terms “sonographer”, “technician”, “viewer”, “operator”, and “practitioner” are used to broadly indicate the person who actively operates the sonography equipment.

The term “highlighting” for a displayed element or feature has its conventional meaning as is understood to those skilled in the information and image display arts. In general, highlighting uses some form of localized display enhancement to attract the attention of the viewer. Highlighting a portion of a display, such as a particular value, graph, message, or other element can be achieved in any of a number of ways, including, but not limited to, annotating, displaying a nearby or overlaying symbol, outlining or tracing, display in a different color or at a markedly different intensity or grayscale value than other image or information content, blinking or animation of a portion of a display, or display at larger scale, higher sharpness, or contrast.

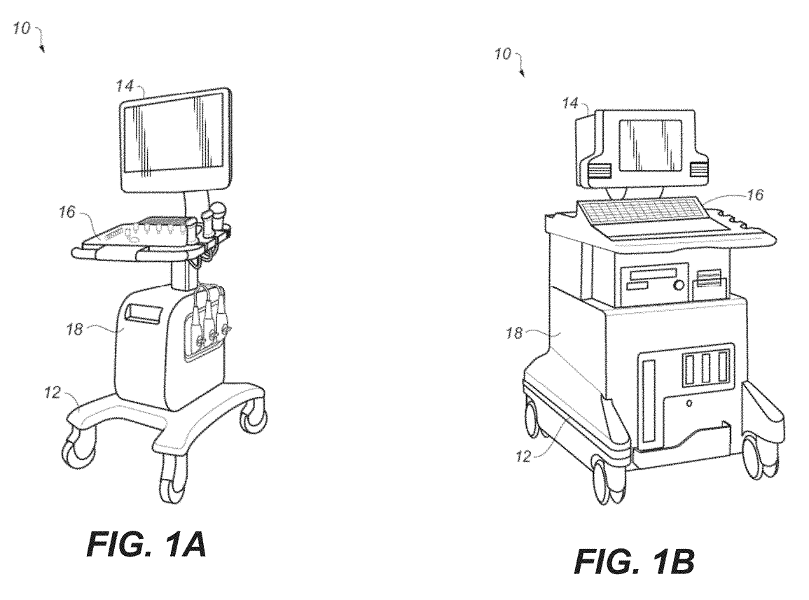

The ultrasound system, shown by way of example in FIGS. 1A and 1B, can include an image processing system, a user interface and a display 14. The image processing system includes a memory and a central processing unit (CPU) or processor. Additional, different, or fewer components may be provided in the system or image processing system. In one embodiment, the system is a medical diagnostic ultrasound imaging system. The memory is a RAM, ROM, hard drive, removable media, compact disc, DVD, floppy disc, tape, cache memory, buffer, capacitor, combinations thereof or any other now known or later developed analog or digital device for storing information. The memory is a storage device operable to store data identifying a selected point or other data for identifying a region of interest. The memory is operable to store data identifying one or a plurality of regions of interest.

With the ultrasound apparatus, information from the user interface indicating a position on an image on the display is used to determine a spatial relationship of a user selected point to a scanned region or image position. The selected point is an individual or single point in one embodiment that may be a point selected within a line, area or volume. Additional or different information may be also stored within the memory. The processor can be a general processor, CPU, application specific integrated circuit, digital signal processor, controller, field programmable gate array, digital device, analog device, arrangement of transistors or other solid-state switches, or combinations thereof, or other now known or later developed devices for receiving analog or digital data and outputting altered or calculated data.

The user input can be a track ball, mouse, joy stick, touch pad, buttons, slider, knobs, position sensor, combinations thereof, or other now known or later developed input devices. The user input is operable to receive an instruction specifying a selected point from a user. For example, the user positions a cursor on an image displayed on the display. The user then selects a position of the cursor as indicating a point for a region of interest. The display can be a CRT, LCD, plasma screen, projector, combinations thereof or other now known or later developed devices for displaying an image, a region of interest, region of interest information, and/or user input information.

Medical ultrasound (also known as diagnostic sonography or ultrasonography) is a diagnostic imaging technique based on the application of ultrasound. It is used to see internal body structures such as tendons, muscles, joints, vessels and internal organs.

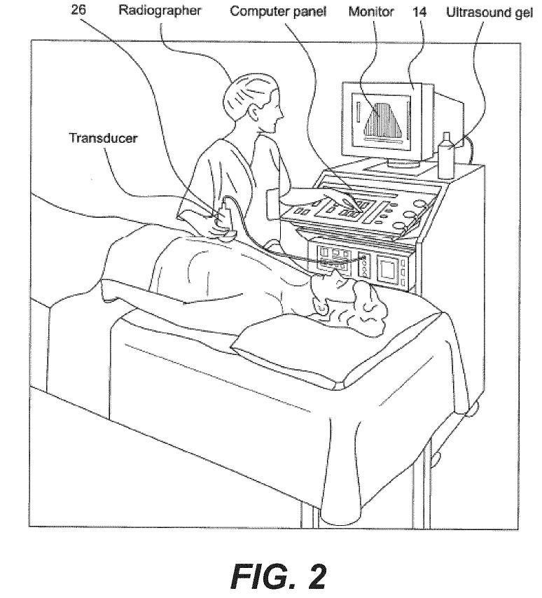

FIGS. 1A-1B and 2-3 show exemplary ultrasound systems 10, each including a cart 12 or other base/support, a display monitor 14, an input interface device (such as keyboard or mouse) 16, and a generator 18. The display can also be a touchscreen to function as an input device. As illustrated, the ultrasound system is a mobile system having wheels.

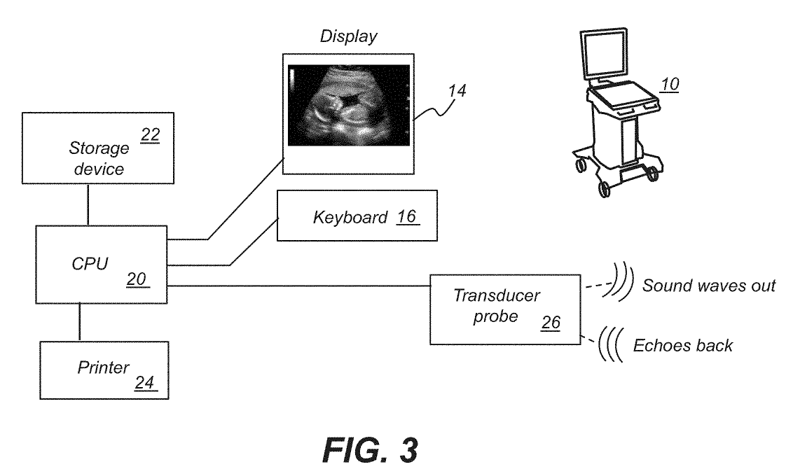

As illustrated, the ultrasound system 10 can be a mobile or portable system designed to be wheeled from one location to another. As FIG. 3 shows, the ultrasound system 10 has a central processing unit CPU 20 that provides control signals and processing capabilities. CPU 20 is in signal communication with display 14 and keyboard or other user interface device 16, as well as with a storage device 22 and an optional printer 24. A transducer probe 26 provides the ultrasound acoustic signal and generates an electronic feedback signal indicative of tissue characteristics according to the echoed sound. FIG. 3 shows an example with an ultrasound image displayed on display monitor 14.

Ultrasound imaging uses sound waves having frequencies higher than those audible to the human ear. Ultrasonic images, also known as sonograms, are made by sending pulses of ultrasound into tissue using a probe. The sound energy echoes off the tissue; with different tissues reflecting varying degrees of sound. These echoes are recorded and displayed as an image to the operator.

Different types of images can be formed using sonographic instruments. The most well-known type is a B-mode image, which displays the acoustic impedance of a two-dimensional cross-section of tissue. Other types of image can display blood flow, motion of tissue over time, the location of blood, the presence of specific molecules, the stiffness of tissue, or the anatomy of a three-dimensional region.

Accordingly, the system of FIGS. 1A-3 is configured to operate within at least two different ultrasound modes. As such, the system provides means to switch between the at least two different ultrasound modes. Such a two-mode configuration and means for switching between modes are well known within the ultrasound technology.

Modes of ultrasound used in medical imaging include: A-mode, B-mode (sometimes referred to as 2D mode), C-mode, M-mode, Doppler mode, Color Doppler (sometimes referred to as Color Flow or color mode), Continuous Doppler, Pulsed wave (PW) Doppler, Duplex: a common name for the simultaneous presentation of 2D and (usually) PW Doppler information. (Using modern ultrasound machines, color Doppler is almost always also used; hence the alternative name Triplex.).

Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.

Harmonic mode: In this mode a deep penetrating fundamental frequency is emitted into the body and a harmonic overtone is detected. This way noise and artifacts due to reverberation and aberration are greatly reduced. Some also believe that penetration depth can be gained with improved lateral resolution; however, this is not well documented.

A sonographer, ultrasonographer, clinician, practitioner, or other clinical user, is a healthcare professional (often a radiographer but may be any healthcare professional with the appropriate training) who specializes in the use of ultrasonic imaging devices to produce diagnostic images, scans, videos, or 3D volumes of anatomy and diagnostic data.

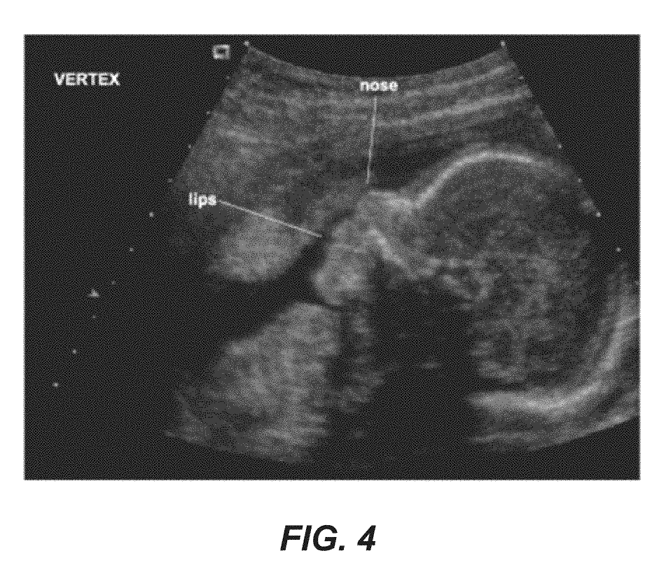

FIG. 4 shows a displayed ultrasound image in grey scale. Such an image would be captured using a grey scale mode, for example, using B-mode.

Applicants have developed a system, method, and interface for an ultrasound medical image. More specifically, there is described a user interface for an ultrasound image measurement, and in particular, for automated and computer-assisted measurements.

### Conventional Workflow for Measurement Acquisition

As noted previously, medical ultrasound imaging systems typically have features related to automatic image interpretation to provide quantitative results to the clinician, sonographer, and other practitioner. Examples of such measurements include automated fetal biometry to measure the anatomical dimensions of various body parts as an indicator of fetal well-being, auto-IMT (intima-media thickness) to measure the distance between the intima and media layers of the internal carotid artery as an indicator of atherosclerosis, and measurements based on cardiac ultrasound.

Image processing and analysis algorithms can be employed to operate in either an automated or semi-automated manner. The workflow implemented as part of these algorithms typically relies on the user selecting a certain location on a displayed image, such as by positioning a cursor, then clicking a mouse or pressing a button on a user console to initiate the measurement. From the Applicants' perspective, this requirement creates an additional undesirable step in the normal clinical workflow, and could distract the user from the core functions of probe positioning and navigating, and interpreting the clinical image.

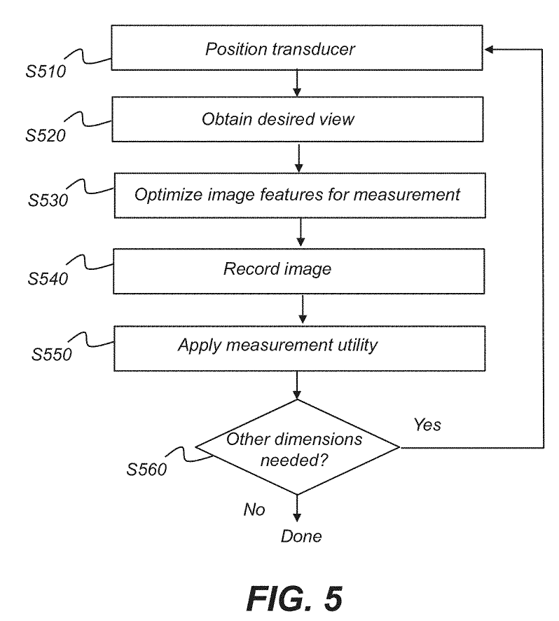

For a better understanding of the present disclosure, it can be useful to consider, in overview, how image measurement is obtained when using conventional ultrasound systems. FIG. 5 shows a sequence that generally applies for a number of different ultrasound systems, with some variation in terms of tools available to the operator and amount of automation provided. In a setup step S510, the sonographer positions the transducer against the patient and begins the process of scanning the anatomy of interest to obtain the desired subject on the display monitor. In a view acquisition step S520, the sonographer locates the desired anatomy and makes any needed position adjustment for obtaining the orientation that is preferred for measurement. For fetal biometry, for example, this can include obtaining the desired relative orientation of face, head, or femur bone or other internal anatomy. For blood flow monitoring, this can include a desired orientation for Doppler imaging of a renal artery or other blood vessel, for example. In a view enhancement step S530, the sonographer can make adjustments that help to heighten contrast, highlight edge features, or otherwise optimize or adapt the image content for improved measurement accuracy. Among image enhancement treatments that can be particularly useful for optimizing measurement accuracy are dynamic range adjustment, color mapping or gray mapping adjustment, gain adjustment, frequency range selection, and filtering.

Continuing with the FIG. 5 process, in an image recording step S540, an operator instruction is entered, indicating that the view that is displayed is considered suitable for initiating recording and measurement functions. Step S540 can be considered as invoking the processes that “freeze” the image for analysis and display. A measurement step S550 then operates upon the recorded image, applying a measurement utility that calculates one or more dimensional measurements that appear on the displayed image and can be recorded. The measurement utility can be a software application or algorithm that operates on the host processor or on some other networked system. A test step S560 determines whether or not additional measurements are to be acquired in the examination for the particular patient. According to an embodiment of the present disclosure, the operator can terminate the measurement process using a command entry mechanism as described subsequently.

As can be appreciated from the flowchart of FIG. 5, there are parts of the imaging process that can depend on the level of operator skill and experience. The task of step S520 for obtaining the view and orientation that works best for acquiring measurement data involves some subjectivity, with results varying from operator to operator as well as from one patient to another. Adjustment of image features for best measurement in step S530 can vary depending on the measurement utilities used by the system as well as on the original image quality obtained.

One particular difficulty with the conventional process of FIG. 5 relates to the mechanics of command entry by the operator. Once the operator has the desired view on display, it is necessary to enter an instruction to record and measure the image content. In some cases, proper positioning requires both hands or positions the operator so that command entry, while maintaining the desired view and orientation of the subject, becomes difficult. In order to maintain the view and orientation of the image to be as stable and consistent as possible, the sonographer must maintain the transducer, held by one hand, in a fixed position, without movement. If movement occurs and the desired view is lost, the sonographer/operator must typically restart the procedure by re-orienting the probe until the desired view is obtained. Concentration on positioning may make it difficult for the sonographer to determine when the obtained view is most acceptable for measurement acquisition and to indicate this to the system.

### Utilities for Improved Sonographer Instruction Entry

Recognizing the above-mentioned difficulties related to obtaining measurements from ultrasound imaging, the Applicants provide apparatus and methods that can help to simplify and automate aspects of operator workflow for the sonographer.

The present disclosure proposes a system and method that employ the use of independent sensors attached to an ultrasound system. The sensors can help to serve the purpose of detecting an operator instruction that indicates when a user would like the automated or semi-automated routines to be invoked.

The Applicants have identified a number of actions which can be employed to (remotely) initiate and conclude measurement during ultrasound scanning, including, but not limited to: (i) gesture detection; (ii) eye tracking; (iii) voice command sensing; (iv) tracking facial expressions or deliberate movement of facial features; (v) foot switch or pedal entry; (vi) pressing a button; and (vii) deliberate change in operator handling of the transducer, such as entering a sequence of finger taps or grip tightening on the transducer (e.g. single tap or long squeeze to initiate measurement, a double tap to conclude).

Actions (i)-(v) above can be considered as hands-free operator actions when sensed and used for instruction entry.

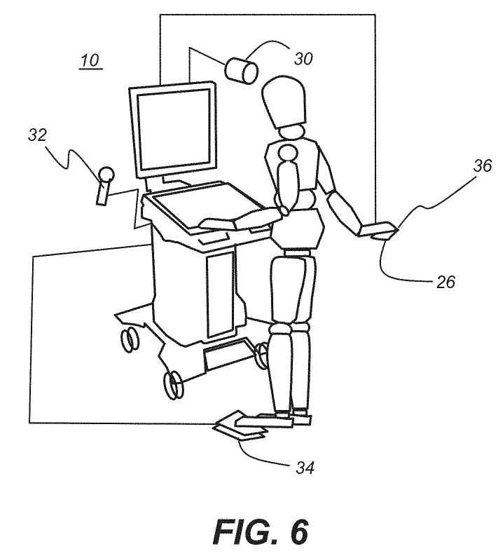

The schematic diagram of FIG. 6 shows ultrasound system 10 with a number of sensors and components that can be used for entry of operator instructions that can initiate image capture in image recording step S540 of FIG. 5. Exemplary sensors that can be used can include a camera 30, a microphone 32, a foot pedal or other foot-operated device 34, or an entry button 36 or other sensor, such as on or near transducer 26 that detects a change in operator handling, as noted in item (vii) above. Hands-free sensors, such as those that detect eye, facial, or other body movement, can be particularly advantageous, allowing the sonographer to hold and maintain transducer placement for ultrasound image capture.

Examples of suitable sensors include, but are not limited to: gesture detectors, voice recognition apparatus, motion detectors, and eye tracking detectors. In one arrangement, when the sensor detects the corresponding stimulus, an internal command is issued within the ultrasound system 10 to launch the automated or semi-automated algorithm for ultrasound image capture and subsequent measurement. In this manner, the user does not need to interrupt clinical scanning procedure or need to manually invoke the algorithm for acquiring and processing measurement; the result is an unobtrusive workflow.

According to an alternate embodiment of the present disclosure, an operator utility can allow customization of instruction entry for using hands-free sensors as described with reference to FIG. 6 or using finger tapping or other deliberate handling of the transducer or other input. A separate software utility can enable the operator to specify measurement response to programmed request actions and to store operator preferences in a separate profile. Upon identification to the ultrasound system, the individual profile can be loaded for the individual sonographer who is logged onto the system for executing an exam session; the profile can include any customized programmable instructions previously entered and stored for the particular sonographer.

A typical workflow is largely sonographer-dependent. That is, for many exam types, there is often no common workflow. Accordingly, employing the workflow of the present disclosure, a sonographer may spend up-front time and effort, entering keystrokes or other instructions in order to obtain the right view, and then launch an automated routine for a measurement. The solutions of the present disclosure relate to methods for employing or embedding automated measurement within the workflow, so that measurement activities can be unobtrusive and the measured results displayed automatically on the screen.

In a further step, the measurement tool, software, and/or algorithm can be re-initiated in order to obtain one or more additional measurements or to repeat the acquisition of measurement dimensions after concluding an intended measurement. Alternately, after all measurements have been acquired, an explicit event may be necessary to end the measurement step. For example, there may be a gesture or equivalent instruction entry to indicate that the desired measurement is complete, and to restore active ultrasound scanning activity.

The use of sensors for instruction entry as mentioned above has been well covered in the literature for a variety of applications, mainly with respect to consumer applications. Applicants' system and method employs the benefits of such sensing for use in a medical ultrasound application.

The system would comprise a software package and either one or multiple sensors that are recognized by the system and integrated with the core ultrasound software. An imaging mode that supports automated measurements is invoked, whereby the trigger detection software will assume a ready or active status waiting to detect the stimulus that would launch the measurement algorithm. When such a stimulus is detected, the software would launch the automated analysis algorithm for measurement.

### Determining Dimensions for Anatomy Features

The dimensional values that can be calculated as part of measurement protocol can vary widely, based on the anatomy of interest, type of exam, age or size of the patient, and other factors. By way of example, and not limitation, just a few of the many biometrics conventionally used for fetal measurement include crown-rump length, head circumference (HC), bi-parietal diameter (BPD), abdominal circumference (AC), and femur length (FL).

Computer-assisted dimensioning provides utilities for generating appropriate dimensional data for various exam types. A typical output of the automated algorithms is a rendering of a shape or multiple shapes on the screen, such as a superimposed line, ellipse, circle, or other bounding feature, or the display of a numeric value, such as on or alongside the displayed image.

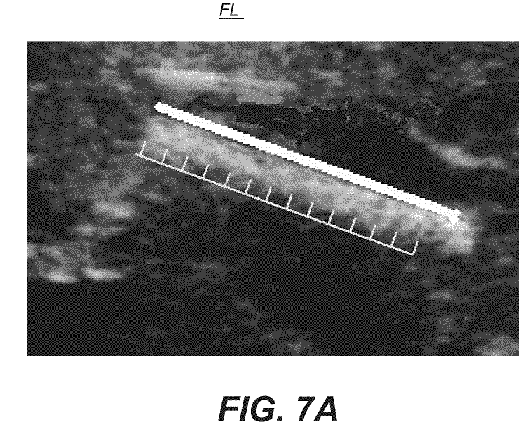

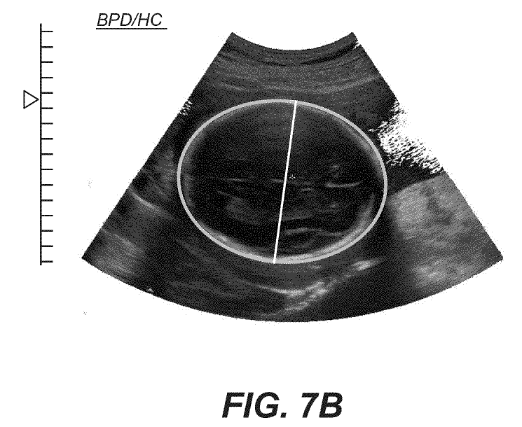

Referring to FIGS. 7A and 7B there are shown exemplary ultrasound images having automated definition of image features of interest and dimensional data. FIG. 7A shows an exemplary femur length (FL) measurement. FIG. 7B shows graphic presentation that can be provided for head circumference (HC) and BPD measurement.

### Computer-Assisted Measurement Method

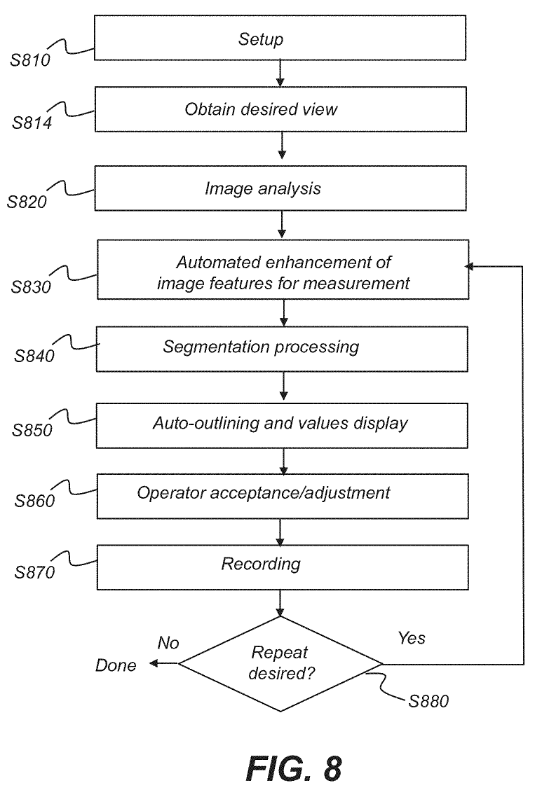

According to an embodiment of the present disclosure, there is provided a computer-assisted measurement method that helps to automate the measurement process. Referring to the logic flow diagram of FIG. 8, the sonographer positions the transducer against the patient and begins the process of scanning the anatomy of interest to obtain the desired subject on the display monitor in a setup step S810, similar to the procedure described with reference to FIG. 5. In addition, the operator specifies the type of measurement to be acquired, such as by entry of an operator instruction. A view acquisition step S814 is also similar to the manual procedure, in which the sonographer locates the desired anatomy and makes any needed position adjustment for obtaining the orientation of the subject that is preferred for measurement. Subsequent processing can optionally be initiated by sensing a gesture or other movement by the sonographer or by accepting a command entered from one of the operator actions (i)-(vii) as listed previously.

Image analysis processing in an image analysis step S820 tracks the image content and, based on previously entered information about the required measurements in setup step S810, automatically detects that the obtained image content is suitable for measurement processing. An auto-enhancement step S830 automatically executes image processing that helps to improve image characteristics to support the desired automatic measurement. A segmentation step S840 processes the image to more accurately identify boundaries and features of the anatomy of interest. An analysis and calculation step S850 then automatically outlines the anatomy of interest and executes the needed dimensional calculations required for the specified exam and displays results to the sonographer. In an operator acceptance step S860, the sonographer accepts or rejects the automated results, which are recorded to memory in a recording step S870 if accepted. A looping step S880 then repeats the processing of steps S830, S840, S850 and S860 (and optionally S820) as many times as needed in order to provide suitable results.

According to an embodiment of the present disclosure, an explicit action by the sonographer can serve as an instruction entry or command to terminate measurement processing, such as following step S870, and restore standard imaging.

As one advantage of repeated image processing, analysis, and calculation, the processing of FIG. 8 can optionally perform an averaging process, updating measured values for step S850 with each iteration that is accepted by the sonographer, thus compensating for minor differences that can occur with operator movement or repositioning of the transducer, as well as for optional signal change or other adjustments made by the operator between image captures. As can be appreciated from the FIG. 8 process, the automated measurement routines allow the sonographer the option to select or reject values, outlining, and views obtained and calculated by the system. In addition, the method allows the operator to select the option to restore manual measurement acquisition, as described previously with reference to FIG. 5.

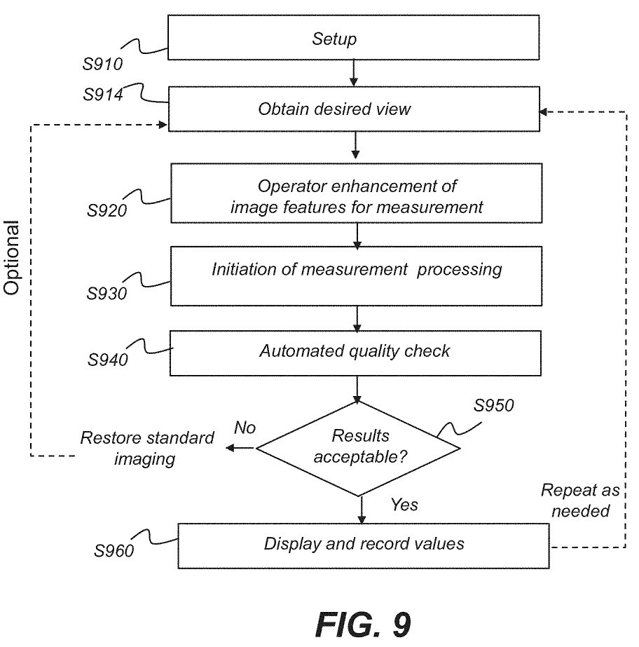

The logic flow diagram of FIG. 9 shows an alternate workflow for a computer-assisted measurement method. The sonographer positions the transducer against the patient and begins the process of scanning the anatomy of interest to obtain the desired subject on the display monitor in a setup step S910, similar to the procedure described with reference to FIG. 5. In addition, the operator specifies the type of measurement to be acquired, such as by entry of an operator instruction. In a view acquisition step S914, the sonographer locates the desired anatomy and makes any needed position adjustment for obtaining the orientation that is preferred for measurement. The sonographer makes any desired adjustments for image quality that are useful for measurement in an enhancement step S920. Measurement processing begins in an initiation step S930. Measurements can be automatically calculated or can be specified by the operator. In a quality check step S940, the system processor then checks the operator adjustments and results, including a comparison of measured results with known patient history (if available) and with stored statistics for a patient population. A decision step S950 can be automated or executed using operator judgment, or can be the result of a computer-assisted process for determining whether or not measurement results are acceptable. Standard imaging can be restored; optionally, the sequence of steps S914, S920, S930, and S940 can be retried. A display and recording step S960 then displays and, optionally, records one or both of image content and measurement values obtained from the exam.

As illustrated in FIG. 5, the workflow would include the steps of: (1) accessing a transducer and placing the transducer on patient; (2) using the transducer, obtaining a desired view and/or orientation of the anatomy of interest (e.g. baby head, abdomen) or physiological blood flow waveform (e.g. renal artery Doppler); (3) optimizing the (displayed) ultrasound image; (4) optionally, freeze the ultrasound image if the measurement protocol so dictates; (5) initiating a measurement tool/software/algorithm using any of the operator actions (e.g. such as indicated in (i)-(vii) above) to obtain a measurement; and (6) concluding the measurement step with any of the operator actions (e.g., such as indicated in the (i)-(vii) listing above); and (7) ending imaging or continuing imaging. The measurement can be displayed, stored, or transmitted.

The present invention can be a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The methods described above may be described with reference to a flowchart. Describing the methods by reference to a flowchart enables one skilled in the art to develop such programs, firmware, or hardware, including such instructions to carry out the methods on suitable computers, executing the instructions from computer-readable media. Similarly, the methods performed by the service computer programs, firmware, or hardware are also composed of computer-executable instructions.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim.

In the following claims, the terms “first,” “second,” and “third,” and the like, are used merely as labels, and are not intended to impose numerical requirements on their objects.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. An ultrasound method, comprising:
using a transducer, obtaining a desired view and/or orientation of the anatomy of interest for an exam;
initiating an automated measurement tool according to sensing of an operator action and obtaining a measurement from a region of interest of the desired view; and
concluding measurement or display according to a sensed operator action.

2. The method of claim 1 further comprising displaying, storing, or transmitting the measurement.

3. The method of claim 1 wherein the sensed operator action is one of the following: a gesture, eye tracking, voice recognition, facial expressions, foot switch entry, pressing a button, squeezing the transducer, and a sequence of taps on the transducer.

4. The method of claim 1 wherein initiating the automated measurement tool is executed automatically.

5. The method of claim 1 further comprising detecting the operator action, and automatically initiating the measurement tool upon detection of the action.

6. The method of claim 1 further comprising re-initiating the measurement tool after concluding the measuring.

7. The method of claim 1 further comprising restoring imaging following the sensed operator action that concludes measurement.

8. The method of claim 1 further comprising enhancing the ultrasound image for measurement.

9. The method of claim 1 further comprising loading operator actions for instruction entry recorded for an individual sonographer.

10. A method for ultrasound measurement, comprising:
obtaining a desired view and orientation of an anatomy of interest;
sensing operator activity to initiate processing and measurement;
enhancing the view for image measurement;
applying segmentation processing to define the anatomy of interest;
outlining the defined anatomy of interest;
calculating a dimension related to the defined anatomy of interest; and
recording the obtained view and calculated dimension.

11. The method of claim 10 wherein sensing the operator activity comprises sensing operator handling of the transducer.

12. A method for ultrasound measurement, comprising:
obtaining a desired view and orientation of the anatomy of interest;
enhancing the view for image measurement according to operator instructions;
applying a measurement processing algorithm to the enhanced view;
executing an automated quality check on results of the measurement processing algorithm; and
displaying or recording results of the measurement processing algorithm.

13. The method of claim 12 further comprising obtaining the operator instructions using hands-free gestures or movements.

14. The method of claim 12 further comprising obtaining the operator instructions using a foot switch or pedal entry.

15. The method of claim 12 further comprising obtaining the operator instructions by handling of a transducer.

16. The method of claim 1 further comprising capturing the ultrasound image according to a measurement protocol for the exam.
